(19) United States
(12) Patent Application Publication
(10) Pub. No.: US 2021/0137467 A1
(43) Pub. Date: May 13, 2021

(54) VITAL INFORMATION DISPLAYING DEVICE, DISPLAY CONTROLLING DEVICE, AND NON-TRANSITORY COMPUTER-READABLE MEDIUM

(71) Applicant: NIHON KOHDEN CORPORATION, Shinjuku-ku, Tokyo (JP)

(72) Inventor: Toshiki AOKI, Tokorozawa-shi, Saitama (JP)

(21) Appl. No.: 16/976,344

(22) PCT Filed: Feb. 15, 2019
(86) PCT No.: PCT/JP2019/005643

§ 371 Date: Aug. 27, 2020

(30) Foreign Application Priority Data
Mar. 1, 2018 (JP) 2018-036670
Dec. 20, 2018 (JP) 2018-238489

(51) Int. Cl. A61B 5/00

(52) U.S. Cl. CPC A61B 5/742; A61B 5/08; A61B 5/7235

(57) ABSTRACT

An input interface is configured to receive a signal corresponding to vital information that exhibits temporal variation. When at least one instruction is executed by a processor, waveform information (W) is generated based on the signal; normalized waveform information (WN) is generated based on the waveform information for a predetermined time period; and temporal variation of the waveform information (W) is displayed on a display section (15) together with the normalized waveform information (WN).

### TECHNICAL FIELD

The presently disclosed subject matter relates to a device for displaying vital information of a subject that exhibits temporal variation. The presently disclosed subject matter also relates to a device for controlling display of the vital information, as well as a non-transitory computer-readable medium having stored a computer program for causing the device to perform the display control.

### BACKGROUND ART

As the vital information of the subject that exhibits temporal variation, a carbon dioxide concentration (partial pressure) in respiration gas, a pulse wave, an electrocardiogram, an electroencephalogram, or the like may be exemplified. Japanese Patent Publication No. 2017-035473A discloses a device for displaying the temporal variation of carbon dioxide concentration in the respiration gas of a subject in the real time manner. The device includes a display area having a vertical axis and a horizontal axis. The vertical axis represents the carbon dioxide concentration value. The horizontal axis represents the elapse of time. As a result, a waveform indicating the temporal variation of the carbon dioxide concentration value is displayed in the display area.

The vital information as described above has a feature that same or similar variation tendency repeatedly appears. For example, in the case of carbon dioxide concentration, the value increases with the expiration of the subject, and decreases with the inspiration. The condition change or abnormality of the subject appears as a remarkable variation of the waveform profile. The medical worker judges the condition change or abnormality of the subject by noting such a variation.

### SUMMARY OF INVENTION

The presently disclosed subject matter is intended to assist judgment of a medical worker based on vital information of a subject that exhibits temporal variation on a display section.

An illustrative aspect of the presently disclosed subject matter provides a vital information displaying device comprising:

an input interface configured to receive a signal corresponding to vital information that exhibits temporal variation;

a processor;

a memory configured to store at least one instruction that is executable by the processor; and

a display section,

wherein when the at least one instruction is executed by the processor,

waveform information is generated based on the signal;

normalized waveform information is generated based on the waveform information for a predetermined time period; and

at least one of temporal variation of the waveform information and chronological change of the normalized waveform information is displayed on the display section together with the normalized waveform information.

An illustrative aspect of the presently disclosed subject matter provides a display controlling device comprising:

an input interface configured to receive a signal corresponding to vital information that exhibits temporal variation;

a processor;

a memory configured to store at least one instruction that is executable by the processor; and

an output interface,

wherein when the at least one instruction is executed by the processor,

waveform information is generated based on the signal;

normalized waveform information is generated based on the waveform information for a predetermined time period; and

a control signal is output from the output interface to cause a display device to display at least one of temporal variation of the waveform information and chronological change of the normalized waveform information together with the normalized waveform information.

An illustrative aspect of the presently disclosed subject matter provides a non-transitory computer-readable medium having stored a computer program including at least one instruction to be executed by a processor of a display controlling device,

wherein when the at least one instruction is executed by the processor,

waveform information is generated based on a signal that is input to an input interface of the display controlling device and corresponds to vital information that exhibits temporal variation;

normalized waveform information is generated based on the waveform information for a predetermined time period; and

a control signal is output from an output interface of the display controlling device to cause a display device to display at least one of temporal variation of the waveform information and chronological change of the normalized waveform information together with the normalized waveform information.

Since the vital information of the subject is not constant, the waveform information displayed on the display section is accompanied by a slight change. If the change in the vital information caused by the condition change or abnormality of the subject is gradual, such change may be absorbed in the normal variations in the waveform information, so that it may be difficult for the medical worker to identify the change. When the waveform information is displayed on the display section together with the normalized waveform information, the medical worker can compare the waveform information generated at any time based on the signal input to the input interface with the normalized waveform information. Since the normalized waveform information can correspond to the steady state of the vital information of the subject, the difference between the normalized waveform information and the temporary change occurring in the waveform information can be made remarkable. Accordingly, the identification of the change in the waveform information due to the condition change or abnormality of the subject can be facilitated to assist the judgment by the medical worker.

When the chronological change of the normalized waveform information is displayed on the display section, it is possible to easily grasp the trend of change in the increasing-decreasing pattern of the carbon dioxide concentration value over a longer time period. Therefore, the identification of a change in the waveform information due to the condition change or abnormality of the subject can be facilitated to assist the judgment by the medical worker.

### BRIEF DESCRIPTION OF DRAWINGS

FIG. 1 illustrates a configuration of a vital information displaying device according to an embodiment.

FIG. 2A illustrates a first method for generating normalized waveform information in the vital information displaying device.

FIG. 2B illustrates the first method for generating normalized waveform information in the vital information displaying device.

FIG. 3A illustrates an operation of a display section in the vital information displaying device.

FIG. 3B illustrates an operation of a display section in the vital information displaying device.

FIG. 4 illustrates a second method for generating normalized waveform information in the vital information displaying device.

FIG. 5A illustrates a third method for generating normalized waveform information in the vital information displaying device.

FIG. 5B illustrates the third method for generating normalized waveform information in the vital information displaying device.

FIG. 6 illustrates the third method for generating normalized waveform information in the vital information displaying device.

FIG. 7 illustrates a configuration of a display controlling device according to an embodiment.

### DESCRIPTION OF EMBODIMENTS

Examples of embodiments are described in detail below with reference to the accompanying drawings. In each drawing, the scale is appropriately changed in order to make each element to be described have a recognizable size.

As illustrated in FIG. 1, a vital information displaying device 1 according to an embodiment includes an input interface 11. A signal corresponding to the vital information of the subject detected through a sensor S is input to the input interface 11. Signal input may be via a wired connection or via wireless communication. The input interface 11 includes a circuit for converting an input signal into data necessary for subsequent processing. As the circuit, an A/D conversion circuit, a filter circuit, or the like may be exemplified.

The vital information displaying device 1 includes a processor 12 and a memory 13. As the processor 12, a CPU, an MPU, a GPU, or the like may be exemplified. The processor 12 may include a plurality of processor cores. As the memory 13, a ROM, a RAM, or the like may be exemplified. The memory 13 may store a computer program for executing processing to be described later. In this case, the memory 13 is an example of the non-transitory computer-readable medium. The computer program may be stored in the memory 13 in advance, or may be downloaded from an external server via a communication network (not illustrated) and the input interface 11. In this case, the external server is an example of the non-transitory computer-readable medium. The computer program may include an artificial intelligence program. As the artificial intelligence program, a neural network trained by deep learning may be exemplified. The computer program is an example of instructions executable by the processor 12. For example, the processor 12 may designate at least a part of the computer program stored in the ROM and load the program on the RAM in order to execute processing described later in cooperation with the RAM.

The processor 12 and the memory 13 are connected to the input interface 11 via a communication bus 14. At least one of the processor 12 and the memory 13 may be provided in a housing independent of the housing in which the input interface 11 is provided, as long as communication with the input interface 11 is possible via a suitable communication interface.

The vital information displaying device 1 includes a display section 15. The display section 15 is connected to the input interface 11, the processor 12, and the memory 13 via the communication bus 14.

In this example, the sensor S detects the carbon dioxide concentration (partial pressure) in the respiration gas of the subject. That is, the sensor S outputs a signal corresponding to the value of the detected carbon dioxide concentration. When the program stored in the memory 13 is executed by the processor 12, waveform information indicating the temporal variation of the carbon dioxide concentration value is generated based on the signal input from the sensor S to the input interface 11.

FIG. 2A illustrates an example of the waveform information W to be generated. The vertical axis represents the value of the carbon dioxide concentration. The horizontal axis represents the elapse of time. Exhalation of the subject increases the carbon dioxide concentration value, whereas inhalation of the subject decreases the carbon dioxide concentration value. Due to repeated respirations of the subject, increasing and decreasing of the carbon dioxide concentration value are repeated alternately. In the drawing, the waveform information W includes patterns P1 to P4 for increasing/decreasing the carbon dioxide concentration value for four respiration cycles (only portions of the pattern P1 and the pattern P4 are illustrated in the drawing).

Next, normalized waveform information WN is generated based on the waveform information W for a predetermined time period. FIG. 2B illustrates a first example of a method of generating the normalized waveform information WN. The predetermined time period T is defined as a time period in which a single increasing-decreasing pattern of the carbon dioxide concentration value can be included.

In the present example, the normalized waveform information WN is obtained by performing the sequential arithmetic averaging process of the increasing-decreasing pattern the carbon dioxide concentration value for every predetermined time period T. Each of the illustrated increasing-decreasing patterns P1 to P4 is a set of carbon dioxide concentration value data obtained in a predetermined sampling period within the predetermined time period T. For example, in a case where the sampling period is (T/100), 100 sampling time points t1 to t100 are included in the predetermined time period T, so that the carbon dioxide concentration value data is assigned to each of the sampling time points.

In the sequential arithmetic averaging process, first, the average value of the carbon dioxide concentration values of the increasing-decreasing patterns P1 and P2 at the sampling time point t1 is obtained. Similarly, the average values of the carbon dioxide concentrations of the increasing-decreasing patterns P1 and P2 at each of the sampling time points t2 to t100 are obtained. As a result, a set of 100 average values are obtained. By arranging these average values in the order of the sampling time points, the normalized waveform information WN is generated.

Subsequently, an average value of the normalized waveform information WN and the carbon dioxide concentration value of the increasing-decreasing pattern P3 at the sampling time point t1 is obtained. Similarly, the average values of the normalized waveform information WN and the average value of the carbon dioxide concentration value of the increasing-decreasing pattern P3 at each of the sampling time points t2 to t100 are obtained. As a result, a set of 100 average values are obtained. By arranging these average values in the order of the sampling time points, new normalized waveform information WN is generated.

The same or similar processing is repeated for the increasing-decreasing patterns P4 and subsequent patterns obtained at any time. Therefore, the normalized waveform information WN is updated every predetermined time period T. As the update is repeatedly performed, the difference between the increasing-decreasing patterns is absorbed, so that the normalized waveform information WN exhibits an increasing-decreasing pattern of the carbon dioxide concentration value corresponding to the steady respiration state of the subject.

Alternatively, the normalized waveform information WN can also be obtained by averaging the increasing-decreasing patterns included in the predetermined time period. For example, when the above-mentioned increasing-decreasing patterns P1 to P4 are included in the predetermined time period, the carbon dioxide concentration value data constituting each increasing-decreasing pattern is temporarily stored in the memory. Subsequently, the average value of the carbon dioxide concentration values of the increasing-decreasing patterns P1 to P4 at each sampling time point is obtained. By arranging these average values in the order of the sampling time points, the normalized waveform information WN is generated. That is, each time the predetermined time period elapses, the normalized waveform information WN is generated and updated based on a plurality of increasing-decreasing patterns included in the predetermined time period.

In the above example, all the increasing-decreasing patterns obtained at any time are used to generate the normalized waveform information WN. However, the normalized waveform information WN may be generated using some of the plurality of obtained increasing-decreasing patterns. Some of the increasing-decreasing patterns may be selected at regular time intervals or at random.

The vital information displaying device 1 is configured to perform characteristic display using the normalized waveform information WN thus obtained.

FIG. 3A illustrates a first display example. Specifically, the display section 15 of the vital information displaying device 1 includes a first display area 51 and a second display area 52. The first display area 51 is an area for displaying the normalized waveform information WN. The second display area 52 is an area for displaying the waveform information W. That is, the waveform information W is displayed on the display section 15 together with the normalized waveform information WN.

In the second display area 52, waveform information W generated at any time based on a signal input to the input interface 11 is displayed. The waveform information W is displayed so as to scroll from the right side to the left side in the second display area 52 with the elapse of time. The normalized waveform information WN displayed in the first display area 51 is updated every predetermined time period T.

Since the respiration of the subject is not constant, the increasing-decreasing pattern of the carbon dioxide concentration value is accompanied by a slight change. If the change in the increasing-decreasing pattern caused by the condition change or abnormality of the subject is gradual, such change may be absorbed in the normal variations in the increasing-decreasing patterns described above, so that it may be difficult for the medical worker to identify the change. According to the above configuration, the medical worker can compare the waveform information W generated at any time based on the signal input to the input interface 11 with the normalized waveform information WN. As described above, since the normalized waveform information WN represents an increasing-decreasing pattern of the carbon dioxide concentration value corresponding to the steady respiration state of the subject, the difference from the temporary change occurring in the waveform information W can be made remarkable. Accordingly, the identification of the change in the waveform information W due to the condition change or abnormality of the subject can be facilitated to assist the judgment by the medical worker.

FIG. 3B illustrates a second display example. In this example, the chronological change of the normalized waveform information WN is displayed on the display section 15. Specifically, each time the normalized waveform information WN is updated a predetermined number of times, new normalized waveform information WN is displayed on the display section 15.

In the illustrated example, the display section 15 includes four display areas. In the leftmost first display area 51, normalized waveform information WN1 that has been updated ten times, for example, is displayed. Next, the normalized waveform information WN2 generated by subjecting the normalized waveform information WN1 to ten times of updates is displayed in the second display area 52 located on the right side of the first display area 51. Similarly, the normalized waveform datum WN3 generated by subjecting the normalized waveform information WN2 to ten times of updates is displayed in the third display area 53 located on the right side of the second display area 52. The normalized waveform information WN4 generated by subjecting the normalized waveform information WN3 to ten times of updated is displayed in the fourth display area 54 located on the right side of the third display area 53.

That is, a normalized waveform information WN generated based on the waveform information W for a predetermined time period within a certain time slot and another normalized waveform information WN generated based on the waveform information W for a predetermined time period within another time slot are displayed on the display section 15 at the same time. Taking the first display area 51 and the second display area 52 as an example, the normalized waveform information WN1 is an example of normalized waveform information generated based on waveform information for a predetermined time period within a first time slot, and the normalized waveform information WN2 is an example of normalized waveform information generated based on waveform information for a predetermined time period within a second time slot.

Further, when a new normalized waveform information WN is generated by subjecting the normalized waveform information WN4 to ten times of updates, the new normalized waveform information WN is displayed in the fourth display area 54. The normalized waveform information WN2, the normalized waveform information WN3, and the normalized waveform information WN4 are displayed in the first display area 51, the second display area 52, and the third display area 53, respectively. Every time new normalized waveform information WN is generated, the old normalized waveform information WN shifts to the display area of the left side.

According to such a configuration, it is possible to easily grasp the trend of change in the increasing-decreasing pattern of the carbon dioxide concentration value over a longer time period. Therefore, the identification of a change in the waveform information W due to the condition change or abnormality of the subject can be facilitated to assist the judgment by the medical worker.

In the above example, the update itself of the normalized waveform information WN is continued. The progress of the update is displayed in each display area. For example, the normalized waveform information WN1 displayed in the first display area 51 is obtained by ten times of updates from a certain time point, and the normalized waveform information WN2 displayed in the second display area 52 is obtained by 20 times of updates from that time point. However, the way of displaying the normalized waveform information WN in the display section 15 is not limited to this example.

For example, each time display on the display section 15 is performed after a predetermined number of updates, generation of the normalized waveform information WN may be restarted. In this instance, the normalized waveform information WN2 displayed in the second display area 52 is generated based on the waveform information W for a predetermined time period obtained from the time point when the normalized waveform information WN1 is generated. Also in this instance, the normalized waveform information WN1 is an example of the normalized waveform information generated based on the waveform information for the predetermined time period in the first time slot, and the normalized waveform information WN2 is an example of the normalized waveform information generated based on the waveform information for the predetermined time period in the second time slot.

In the above example, the waveform information W for generating the normalized waveform information WN to be displayed in one of the adjacent display areas and the waveform information W for generating the normalized waveform information WN to be displayed in the other display area are continuous in time. However, the waveform information W for generating the normalized waveform information WN to be displayed in each of the adjacent display areas need not be continuous in time.

For example, the normalized waveform information WN1 displayed in the first display area 51 may be generated based on the waveform information W for the predetermined time period that was obtained 40 minutes ago, the normalized waveform information WN2 displayed in the second display area 52 may be generated based on the waveform information W for the predetermined time period that was obtained 30 minutes ago, the normalized waveform information WN3 displayed in the third display area 53 may be generated based on the waveform information W for the predetermined time period that was obtained 20 minutes ago, and the normalized waveform information WN4 displayed in the fourth display area 54 may be generated based on the waveform information W for the predetermined time period that was obtained 10 minutes ago. Also in this instance, the normalized waveform information WN1 is an example of the normalized waveform information generated based on the waveform information for the predetermined time period in the first time slot, and the normalized waveform information WN2 is an example of the normalized waveform information generated based on the waveform information for the predetermined time period in the second time slot.

In the above example, the interval between the time points when the waveform information W for generating the normalized waveform information WN to be displayed in each of the adjacent display areas are obtained is constant. However, the interval need not be constant.

For example, the normalized waveform information WN1 displayed in the first display area 51 may be generated based on the waveform information W for the predetermined time period that was obtained one day ago, the normalized waveform information WN2 displayed in the second display area 52 may be generated based on the waveform information W for the predetermined time period that was obtained six hours ago, the normalized waveform information WN3 displayed in the third display area 53 may be generated based on the waveform information W for the predetermined time period that was obtained one hour ago, and the normalized waveform information WN4 displayed in the fourth display area 54 may be generated based on the waveform information W for the predetermined time period that was obtained one minute ago. Also in this instance, the normalized waveform information WN1 is an example of the normalized waveform information generated based on the waveform information for the predetermined time period in the first time slot, and the normalized waveform information WN2 is an example of the normalized waveform information generated based on the waveform information for the predetermined time period in the second time slot.

That is, the normalized waveform information WN generated at an arbitrary time point can be displayed in each display area. However, it is preferable that the normalized waveform information WN to be displayed on the display section 15 are arranged in the order of generation, for example, from left side to right side.

FIG. 4 illustrates a second example of the method of generating the normalized waveform information WN. In this example, only the waveform information W whose difference from the reference waveform is within a predetermined range is subjected to arithmetic averaging process to generate the normalized waveform information WN.

First, an increasing-decreasing pattern P1 of the carbon dioxide concentration value included in the waveform information W is set as the reference waveform. Then, the increasing-decreasing pattern P2 of the carbon dioxide concentration value is subjected to comparison with the reference waveform. A boundary B indicated by dashed lines around the increasing-decreasing pattern P2 represents the “predetermined range” described above. Specifically, a predetermined range (e.g., plus or minus 5 mmHg) is defined with reference to the carbon dioxide concentration values at the respective sampling time points in the reference waveforms, so that a set of the ranges forms the boundary B.

In the illustrated example, the entire increasing-decreasing pattern P2 is located within the boundary B. In this case, the arithmetic averaging process of the increasing-decreasing pattern P2 with respect to the reference waveform (the increasing-decreasing pattern P1) is permitted. The arithmetic averaging process is performed in accordance with the method described referring to FIG. 2B, so that a normalized waveform information WN1 is generated.

Next, the normalized waveform data WN1 is set as the reference waveform. The extent of the border B is updated based on the normalized waveform information WN1. Then, the increasing-decreasing pattern P3 of the carbon dioxide concentration value is subjected to comparison with the reference waveform.

The increasing-decreasing pattern P3 is also entirely located within the boundary B. In this instance, the arithmetic averaging process of the increasing-decreasing patterns P3 with respect to the reference waveform (the normalized waveform data WN1) is permitted. When the averaging process is performed, the normalized waveform information WN2 is generated.

Next, the normalized waveform information WN2 is set as the reference waveform. The extent of the border B is updated based on the normalized waveform information WN2. The increasing-decreasing pattern P4 of the carbon dioxide concentration value is subjected to comparison with the reference waveform.

A portion of the increasing-decreasing pattern P4 is located outside the boundary B. In this instance, it is determined that the difference from the reference waveform is not within the predetermined range, so that the arithmetic averaging process of the increasing-decreasing patterns P4 with respect to the reference waveform (the normalized waveform data WN2) is not permitted. Therefore, the normalized waveform information WN2 is maintained. Thereafter, the same processing is repeated.

The spike-like variation appeared in the increasing-decreasing pattern P4 may occur suddenly due to a coughing, a conversation, a position change, or the like of the subject. According to the above configuration, such sudden variations can be avoided from being subjected to the arithmetic averaging process. Therefore, it is possible to reduce noise that can be included in the normalized waveform information WN used for judging the condition change or abnormality of the subject.

On the other hand, when the number of increasing-decreasing patterns for which the arithmetic averaging process is not permitted is relatively large, it can be said that there is a high possibility that another factor is involved in a phenomenon in which a portion of the increasing-decreasing patterns is located outside the boundary B. In this case, it can be said that the reliability of the obtained normalized waveform information WN is rather lowered. Accordingly, the processor 12 may be configured to obtain a ratio of the increasing-decreasing patterns that have not been permitted to be subjected to the arithmetic averaging process to the increasing-decreasing patterns that have been permitted to be subjected to the arithmetic averaging process, and to perform notification when the ratio exceeds a threshold value. A new parameter, such as a reliability corresponding to the ratio, may be generated.

According to such a configuration, it is possible to recognize the possibility that the difference between the increasing-decreasing pattern and the reference waveform is increased due to a cause other than noise.

In this example, the normalized waveform information WN is generated by using the arithmetic averaging process. However, the normalized waveform information WN may be generated using the weighted averaging process. For example, an averaging process may be performed in which the weighting coefficient is increased as the value included in the waveform information W is closer to the carbon dioxide concentration value included in the reference waveform. The normalized waveform information WN may be generated using the harmonic averaging process or the geometric averaging process.

Next, a third example of a method of generating the normalized waveform information WN will be described. In this example, the normalized waveform information WN is generated by performing statistical processing on a plurality of profile factors of the waveform information W.

The “profile factor” is a variety of parameters that characterize the shape of the increasing-decreasing pattern of the carbon dioxide concentration value. Details of each profile factor illustrated in FIG. 5A are as follows.
[theta]r: rising angle (gradient of a portion where the concentration value steeply increases)

[theta]p: top portion angle (gradient of the portion where the concentration value gently increases near the peak value)

[theta]f: falling angle (gradient of a portion where the concentration value steeply decreases)

Vr: rising velocity (rate of change at the portion where the concentration value steeply increases)

Vp: top portion velocity (rate of change at the portion where the concentration value gently increases near the peak value)

Vf: falling velocity (rate of change at the portion where the concentration value steeply decreases)

Vra: absolute value of Vr

Vpa: absolute value of Vp

Vfa: absolute value of Vf

Trf: rising-falling time interval (time interval from the start of increasing to the end of decreasing in the concentration value)

Tfr: falling-rising time interval (the time interval from the end of the decreasing in the concentration value of one increasing-decreasing pattern to the start of the increasing in the concentration value of the next increasing-decreasing pattern)

Trr: rising-rising time interval (time interval from the start of increasing in concentration value of one increasing-decreasing pattern to the start of increasing in concentration value of the next increasing-decreasing pattern)

Tff: falling-falling time interval (the time interval from the end of the decreasing in the concentration value of one increasing-decreasing pattern to the end of the decreasing in the concentration value of the next increasing-decreasing pattern)

A: under-waveform area (area of the area surrounded by a baseline indicating a reference concentration value and the waveform)

The “portion where the concentration value steeply increases” can be defined as, for example, a portion where the concentration value increases from 10% to 90% of the peak value. The “portion where the concentration value gently increases” can be defined as, for example, a portion where the concentration value increases from 90% of the peak value to the peak value. The “portion where the concentration value steeply decreases” can be defined as a portion where the concentration value decreases from 90% to 10% of the peak value, for example. The “start of increasing in the concentration value” may be defined, for example, as the time point at which the increasing concentration value reaches 10% of the peak value. The “end of decreasing of the concentration value” can be defined as, for example, a time point when the decreasing concentration value reaches 10% of the peak value.

Alternatively, by obtaining the differential value (variation speed) of the variation in the concentration value, the “portion where the concentration value steeply increases”, the “portion where the concentration value gently increases”, and the “portion where the concentration value steeply decreases” may be defined. For example, a time period during which the obtained differential value is greater than a predetermined positive threshold value may be defined as a “portion where the concentration value steeply increases”. Similarly, a time period during which the differential value is no greater than the predetermined positive threshold value may be defined as a “portion where the concentration value gently increases”, and a time period during which the differential value is less than a predetermined negative threshold value may be defined as a “portion where the concentration value steeply decreases”.

When the normalized waveform information WN is generated, an appropriate combination of factors is selected from the above-listed profile factors. For example, a combination of the rising angle [theta]r, the top portion angle [theta]p, the falling angle [theta]f, and the rising-falling time interval Trf may be selected.

As illustrated in FIG. 5B, each time an increasing-decreasing pattern of the carbon dioxide concentration value appears in the waveform information W generated at any time based on the signal input from the sensor S to the input interface 11, the rising angle [theta]r, the top portion angle [theta]p, the falling angle [theta]f, and the rising-falling time interval Trf are obtained. Average values are then obtained for the respective profile factors ([theta]rm, [theta]pm, [theta]fm, Tfrm). Each of the obtained average values is set as a value of a profile factor of the increasing-decreasing pattern of the carbon dioxide concentration value in the normalized waveform information WN.

FIG. 6 illustrates the distribution of the profile factors obtained as described above. In the drawing, only the rising angle [theta]r and the rising-falling time interval Trf are illustrated. In this example, only the value of the profile factor whose difference from the average value is within a predetermined range is used for generating the normalized waveform information WN. That is, only the value of the profile factor distributed between the two dashed lines in the drawing is used to generate the normalized waveform information WN.

Even with such a manner, it is possible to avoid a sudden variation in the waveform information W that may occur due to coughing, conversation, posture change, or the like of the subject from being used to generate the normalized waveform information WN. Therefore, it is possible to reduce noise that can be included in the normalized waveform information WN used for judging the condition change or abnormality of the subject.

On the other hand, when the number of profile factors that are not used for generating the normalized waveform information WN is relatively large, it is highly likely that another factor is involved in the phenomenon that the difference between the value of the profile factor and the average value becomes large. In this case, it is necessary to consider the reliability of the obtained normalized waveform information WN. Accordingly, the processor 12 may be configured to obtain the ratio of the profile factor that has not been used for generating the normalized waveform information WN to the profile factor that has been used for generating the normalized waveform information WN, and to notify when the ratio exceeds a threshold value. A new parameter, such as a reliability corresponding to the ratio, may be generated.

According to such a configuration. it is possible to recognize the possibility that the difference between the profile factor and the average value is increased due to a cause other than noise.

The value used as the profile factor of the normalized waveform information WN is not limited to the average value. A median value, a mode value, or the like may be used in accordance with the distribution of the profile factor obtained from the waveform information W. The average value, the median value, and the mode value are examples of a statistic representative value.

The above embodiment is merely exemplary to facilitate understanding of the presently disclosed subject matter. The configuration according to the above embodiment can be appropriately modified or improved without departing from the fundamental concept of the presently disclosed subject matter.

In the above embodiment, the processor 12 for generating the normalized waveform information WN and the display section 15 for displaying the normalized waveform information WN are provided in the same device. However, the generation and display of the normalized waveform information WN may be performed by independent devices.

FIG. 7 illustrates a display controlling device 2 capable of realizing such an operation. Components substantially the same as those of the vital information displaying device 1 illustrated in FIG. 1 are denoted by the same reference numerals. Repetitive descriptions for those will be omitted.

The display controlling device 2 may be connected to a display device 3 via a communication network. The display controlling device 2 includes an output interface 21. The processor 12 may cause the output interface 21 to output a control signal for causing the display device 3 to display the generated normalized waveform information WN.

In the above embodiment, the carbon dioxide concentration (partial pressure) in the respiration gas is exemplified as vital information of the subject. However, the above configuration can be applied to the display of the vital information that exhibits temporal variation while the same trend of variations repeatedly appears. As such vital information, concentration (partial pressure) of oxygen or anesthetic gas in the respiration gas, a flow of the respiration gas, a volume of the respiration gas, pulse wave, electrocardiogram (ECG), and electroencephalogram (EEG).

In the embodiment described above, the waveform information W is displayed in the area formed by the horizontal axis representing the elapse of time and the vertical axis representing the measured value (carbon dioxide concentration value). However, the waveform information W may be displayed in an area where both the horizontal axis and the vertical axis represent the measured value. For example, the waveform information W may be displayed in an area formed by the horizontal axis representing the ventilation amount and the vertical axis representing the carbon dioxide concentration value. Alternatively, the waveform information W may be displayed in an area formed by the horizontal axis representing the airway internal pressure and the vertical axis representing the ventilation amount. Alternatively, the waveform information W may be displayed in an area formed by the horizontal axis representing the ventilation amount and the vertical axis representing the ventilation flow rate. As for such waveform information W, normalized waveform information WN can be generated every predetermined time period as well.

The present application is based on Japanese Patent Application No. 2018-036670 filed on Mar. 1, 2018 and Japanese Patent Application No. 2018-238489 filed on Dec. 20, 2018, the entire contents of which are hereby incorporated by reference.

1. A vital information displaying device comprising:
an input interface configured to receive a signal corresponding to vital information that exhibits temporal variation;
a processor;
a memory configured to store at least one instruction that is executable by the processor; and
a display section,
wherein when the at least one instruction is executed by the processor,
waveform information is generated based on the signal;
normalized waveform information is generated based on the waveform information for a predetermined time period; and
at least one of temporal variation of the waveform information and chronological change of the normalized waveform information is displayed on the display section together with the normalized waveform information.

2. The vital information displaying device according to claim 1, wherein the normalized waveform information is generated by subjecting only the waveform information whose difference from a reference waveform is within a predetermined range to averaging process with respect to the reference waveform.

3. The vital information displaying device according to claim 2, wherein a ratio of the waveform information that have not been subjected to the averaging process to the waveform information that have been subjected to the averaging process is obtained.

4. The vital information displaying device according to claim 1, wherein the normalized waveform information is generated by performing statistical processing with respect to a plurality of profile factors of the waveform information.

5. The vital information displaying device according to claim 4, wherein the normalized waveform information is generated by subjecting only the profile factor whose difference from a statistic representative value is within a predetermined range to the statistical processing.

6. The vital information displaying device according to claim 5, wherein a ratio of the waveform information that have not been subjected to the statistical processing to the waveform information that have been subjected to the statistical processing is obtained.

7. The vital information displaying device according to claim 1, wherein the normalized waveform information generated based on the waveform information for the predetermined time period within a first time slot and the normalized waveform information generated based on the waveform information for the predetermined time period within a second time slot that is different from the first time slot are displayed on the display section at the same time.

8. A display controlling device comprising:
an input interface configured to receive a signal corresponding to vital information that exhibits temporal variation;
a processor;
a memory configured to store at least one instruction that is executable by the processor; and
an output interface,
wherein when the at least one instruction is executed by the processor,
waveform information is generated based on the signal;
normalized waveform information is generated based on the waveform information for a predetermined time period; and
a control signal is output from the output interface to cause a display device to display at least one of temporal variation of the waveform information and chronological change of the normalized waveform information together with the normalized waveform information.

9. A non-transitory computer-readable medium having stored a computer program including at least one instruction to be executed by a processor of a display controlling device,
wherein when the at least one instruction is executed by the processor,
waveform information is generated based on a signal that is input to an input interface of the display controlling device and corresponds to vital information that exhibits temporal variation;
normalized waveform information is generated based on the waveform information for a predetermined time period; and
a control signal is output from an output interface of the display controlling device to cause a display device to display at least one of temporal variation of the waveform information and chronological change of the normalized waveform information together with the normalized waveform information.
